(19) United States
(12) Patent Application Publication
(10) Pub. No.: US 2001/0018052 A1
(43) Pub. Date: Aug. 30, 2001

(54) Use of anti-coagulation factor antibodies as long-lasting protective agents

(75) Inventor: Giora Z. Feuerstein, Wynnewood, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 09/817,960

(22) Filed: Mar. 27, 2001

Related U.S. Application Data
(63) Continuation of application No. 09/359,202, filed on Jul. 22, 1999, now abandoned.
(60) Provisional application No. 60/095,714, filed on Aug. 7, 1998.

(51) Int. Cl. A61K 39/395

(52) U.S. Cl. 424/145.1

(57) ABSTRACT

The use of antibodies and antigen-binding fragments directed against coagulation factors and their use in inhibiting thrombosis are disclosed.

This application claims the benefit of U.S. Provisional Application No. 60/095,714, filed Aug. 7, 1998.

### FIELD OF THE INVENTION

This invention relates to the use of antibodies and antigen-binding fragments that bind to coagulation factors as long-lasting inhibitors of thrombosis.

### BACKGROUND OF THE INVENTION

Under normal circumstances, an injury, be it minor or major, to vascular endothelial cells lining a blood vessel triggers a hemostatic response through a sequence of events commonly referred to as the coagulation “cascade.” The cascade culminates in the conversion of soluble fibrinogen to insoluble fibrin which, together with platelets, forms a localized clot or thrombus which prevents extravasation of blood components. Wound healing can then occur followed by clot dissolution and restoration of blood vessel integrity and flow.

The events which occur between injury and clot formation are a carefully regulated and linked series of reactions. In brief, a number of plasma coagulation proteins in inactive proenzyme forms and cofactors circulate in the blood. Active enzyme complexes are assembled at an injury site and are sequentially activated to serine proteases, with each successive serine protease catalyzing the subsequent proenzyme to protease activation. This enzymatic cascade results in each step magnifying the effect of the succeeding step. For an overview of the coagulation cascade see the first chapter of “Thombosis and Hemorrhage”, J. Loscalzo and A. Schafer, eds., Blackwell Scientific Publications, Oxford, England (1994).

While efficient clotting limits the loss of blood at an injury site, inappropriate formation of thrombi in veins or arteries is a common cause of disability and death. Abnormal clotting activity can result in and/or from pathologies or treatments such as myocardial infarction, unstable angina, atrial fibrillation, stroke, renal damage, percutaneous translumenal coronary angioplasty, disseminated intravascular coagulation, sepsis, pulmonary embolism and deep vein thrombosis. The formation of clots on foreign surfaces of artificial organs, shunts and prostheses such as artificial heart valves is also problematic.

Approved anticoagulant agents currently used in treatment of these pathologies and other thrombotic and embolic disorders include the sulfated heteropolysaccharides heparin and low molecular weight (LMW) heparin. These agents are administered parenterally and can cause rapid and complete inhibition of clotting by activation of the thrombin inhibitor, antithrombin III and inactivation of all of the clotting factors. However, a lack of durability of treatment effect has been shown for heparin and other thrombin inhibitors. Heparin's antithrombotic effect is observed only while a patient is on the drug. Further, discontinuation of heparin therapy can produce a rebound effect where patients become hypercoagulable and have an increased incidence of cardiac events. See Becker et al. in Am. Heart J. 131, 421-433 (1996) and Granger et al. in Am. Heart J. 93, 870-878 (1996). Clearly, a need exists for an anticoagulant agent which has a long-lasting duration of efficacy in controlling thrombotic and embolic disorders.

### SUMMARY OF THE INVENTION

Accordingly, an aspect of the present invention is a method for inhibiting thrombosis in an animal comprising administering an effective dose of an anti-coagulation factor antibody having long-lasting protective activity.

### BRIEF DESCRIPTION OF THE DRAWINGS

FIG. 1 is a histogram of experimental results demonstrating the effect of heparin on thrombus weight and carotid blood flow in a rat carotid thrombosis model 3 and 7 days after dosing.

FIG. 2 is a histogram of experimental results demonstrating the effect of a 9 mg/kg bolus injection of anti-Factor IX mAb on thrombus weight and cartoid blood flow in a rat carotid thrombosis model 3 and 7 days after dosing.

FIG. 3 is a histogram of experimental results demonstrating the effect of two 6 mg/kg bolus injections of anti-Factor IX mAb on thrombus weight and cartoid blood flow in a rat carotid thrombosis model 7 days after dosing.

FIG. 4 is a histogram of experimental results demonstrating the effect of two 6 mg/kg bolus injections of anti-Factor IX mAb on thrombus weight and cartoid blood flow in a rat carotid thrombosis model 30 days after dosing.

FIG. 5 is a graph of experimental results demonstrating the effects of a 1 or 3 mg intravenous bolus followed by a 0.3 or 1 mg/kg 75 min infusion on cerebral cortex microvascular infusion in aniamls treated with a middle cerebral artery occlusion/thrombus(MCAO)-inducing FeCl₃ micropatch application.

FIG. 6 is a histogram of experimental results demonstrating the effects of a 1 or 3 mg intravenous bolus followed by a 0.3 or 1 mg/kg 75 min infusion on cerebral cortex hemispheric infarction 24 hours later in the same animals as FIG. 5.

FIG. 7 is a histogram of experimental results demonstrating the effects of a 1 or 3 mg intravenous bolus followed by a 0.3 or 1 mg/kg 75 min infusion on cerebral cortex infarct volume 24 hours later in the same animals as in FIGS. 5 and 6.

FIG. 8 is a histogram of experimental results demonstrating the effects of a 1 or 3 mg intravenous bolus followed by a 0.3 or 1 mg/kg 75 min infusion on forelimb neurological deficit (representing degree of contralateral forelimb paralysis and weakness 24 hours later in the same animals as in FIGS. 5-7.

FIG. 9 is a histogram of experimental results demonstrating the effects of a 1 or 3 mg intravenous bolus followed by a 0.3 or 1 mg/kg 75 min infusion on hindlimb neurological deficit (representing degree of abnormal hindlimb placement 24 hours later in the same animals as FIGS. 5-8.

### DETAILED DESCRIPTION OF THE INVENTION

All publications, including but not limited to patents and patent applications, cited in this specification are herein incorporated by reference as though fully set forth.

As used herein, the term “antibody” refers to immunoglobulins which can be prepared by hybridoma techniques, phage display combinatorial libraries, immunoglobulin chain shuffling, humanization and other recombinant techniques. Also included are fully human monoclonal antibodies. “Antibody” also includes proteins encoded by an altered immunoglobulin coding region which may be obtained by humanization, fragmenting, engineering by recombinant DNA methods or by chemical modifications. Such altered immunoglobulins include humanized, primatized or chimeric antibodies or antigen-binding fragments, e.g., Fv, Fab, Fab′ or F(ab′)₂ and the like.

As used herein, the term “long-lasting protective activity” refers to the activity of an antibody that binds to a human coagulation factor, preferably from the intrinsic and common pathways, including Factor IX/IXa, X/Xa, XI/XIa, VIII/VIIIa and V/Va, VII/VIIa and thrombin and has a duration of clinical effect that is longer than heparin or other thrombin inhibitors such as low molecular weight heparin and warfarin. “Duration of clinical effect” is defined as the time period that an antithrombotic effect is observed. Preferably, the duration of clinical effect is up to 30 days.

The present invention provides for the use of a variety of antibodies and antigen-binding fragments thereof directed against coagulation factors, which are characterized by long-lasting protective activity. The invention relates to a method for inhibiting thrombosis in an animal, particularly a human, which comprises administering an effective dose of an anti-coagulation factor antibody having long-lasting protective activity. Preferably, the coagulation factor is from the intrinsic or common coagulation pathway. Most preferably, the anti-coagulation factor antibodies are anti-Factor IX, anti-Factor IXa, anti-Factor X, anti-Factor Xa, anti-Factor XI, anti-Factor XIa, anti-Factor VIII, anti-Factor VIIIa, anti-Factor V, anti-Factor Va, anti-Factor VII, anti-Factor VIIa or anti-thrombin. Particularly preferred are anti-Factor IX antibodies.

Exemplary anti-coagulation factor antibodies useful in the method of the invention are the humanized monoclonal antibodies SB 249413, SB 249415, SB 249416, SB 249417, SB 257731 and SB 257732 directed against human Factor IX, the chimeric monoclonal antibody chαFIX directed against human Factor IX, the murine monoclonal antibodies BC1, BC2, 9E4(2)F4 and 11G4(1)B9 which are directed against human Factor IX and/or Factor IXa or the murine monoclonal antibodies HFXLC and HFXI which are directed against human Factors X and XI, respectively and antigen-binding fragments thereof. Particularly preferred is the anti-human Factor IX monoclonal antibody SB 249417. Also preferred is a Fab fragment of SB 249417. All of the exemplary antibodies listed above are disclosed in U.S. patent application Ser. No. 08/783,853.

Alternatively, anti-platelet agents such as acetylsalicylic acid can be administered in combination with the anti-coagulation factor monoclonal antibody. In some cases, combination therapy lowers the therapeutically effective dose of anti-coagulation factor monoclonal antibody.

The methods of the invention are useful in therapy of thrombotic and embolic disorders such as those associated with myocardial infarction, unstable angina, atrial fibrillation, stroke, renal damage, pulmonary embolism, deep vein thrombosis, percutaneous translumenal coronary angioplasty, disseminated intravascular coagulation, sepsis, artificial organs, shunts or prostheses.

The antibodies and antigen-binding fragments of this invention may also be used in conjunction with other antibodies, particularly antibodies reactive with other markers (epitopes) responsible for the condition against which the antibody of the method of the invention is directed.

The method of the invention is desirable for treatment of abnormal clotting conditions from about 1 day to about 30 days, or as needed. This represents a considerable advance over the currently used anticoagulants heparin and warfarin. The length of treatment relates to the relative duration of the clinical effect of protective activity and can be adjusted by one of skill in the art depending upon the condition being treated and the general health of the patient.

The mode of administration of the antibody or antigen-binding fragment useful in the invention may be any suitable route which delivers the agent to the host. The antibodies, antigen-binding fragments and pharmaceutical compositions thereof are particularly useful for parenteral administration, i.e., subcutaneously, intramuscularly, intravenously or intranasally.

Therapeutic agents of the invention may be prepared as pharmaceutical compositions containing an effective amount of antibody or antigen-bindiing fragment as an active ingredient in a pharmaceutically acceptable carrier. An aqueous suspension or solution containing the antibody or antigen-binding fragment, preferably buffered at physiological pH, in a form ready for injection is preferred. The compositions for parenteral administration will commonly comprise a solution of the antibody or antigen-binding fragment or a cocktail thereof dissolved in an pharmaceutically acceptable carrier, preferably an aqueous carrier. A variety of aqueous carriers may be employed, e.g., 0.4% saline, 0.3% glycine and the like. These solutions are sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the antibody of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition useful in the method of the invention for intramuscular injection could be prepared to contain 1 mL sterile buffered water, and between about 1 ng to about 100 mg, e.g. about 50 ng to about 30 mg or more preferably, about 5 mg to about 25 mg, of an antibody or antigen-binding fragment. Similarly, a pharmaceutical composition of the invention for intravenous infusion could be made up to contain about 250 ml of sterile Ringer's solution, and about 1 mg to about 30 mg and preferably 5 mg to about 25 mg of an engineered antibody of the invention. Actual methods for preparing parenterally administrable compositions are well known or will be apparent to those skilled in the art and are described in more detail in, for example, “Remington's Pharmaceutical Science”, 15th ed., Mack Publishing Company, Easton, Pa.

It is preferred that the therapeutic agent used in the method of the invention, when in a pharmaceutical preparation, be present in unit dose forms. The appropriate therapeutically effective dose can be determined readily by those of skill in the art. To effectively treat a thrombotic or embolic disorder in a human or other animal, one dose of approximately 0.1 mg to approximately 20 mg per kg body weight of antibody or antigen-binding fragment should be administered parenterally, preferably i.v. or i.m. Such dose may, if necessary, be repeated at appropriate time intervals selected as appropriate by a physician during the thrombotic response.

The present invention will now be described with reference to the following specific, non-limiting examples.

### EXAMPLE 1

### Passivation Effect of Anti-Factor IX Monoclonal Antibodies

In order to evaluate the duration of efficacy of anti-Factor IX antibodies in prevention of arterial thrombosis, the rat carotid artery thrombosis model as reported by Schumacher et al. in J. Cardio. Pharm. 22, 526-533 (1993) was adapted. This model consists of segmental injury to the carotid endothelium by oxygen radicals generated by FeCl₃ solution applied on the surface of the carotid artery.

In brief, male rats (Charles River Raleigh, 350-490 g) were anesthetized with sodium pentobarbital at 50 mg/kg i.p. Using aseptic procedure, the rats were placed dorsally on a heated surgical board. An incision was made in the neck area, the left carotid artery isolated and a piece of parafilm M (1×4 mm) placed under the artery. A cannula was inserted in the tail vein for vehicle or drug administration. A premeasured 6 mm diameter fiberglass patch soaked in 50% FeCl₃ solution was placed on the carotid artery for 10 minutes, removed and the animals observed for 20 minutes. The incision was cleaned and sutured and the animals placed in a heated cage to recover. Three, seven or thirty days after carotid injury, the rat was anesthetized, the incision opened and the carotid isolated for observation of blood flow. The carotid thrombus was extracted from the carotid artery and weighed.

SB249415 was administered 15 minutes prior to the onset of carotid injury. Heparin-treated animals were infused for 1 hour prior to injury. The following treatments were examined.

1. Heparin: 120 U/kg bolus, followed by infusion of 4 U/kg/min over 60 minutes

2. Anti-Factor IX mAb SB249415: 6 or 9 mg/kg bolus

3. Anti-Factor IX mAb SB249415: 6 mg/kg bolus on Day 0 followed by 6 mg/kg bolus on Day 1 (24 h later)

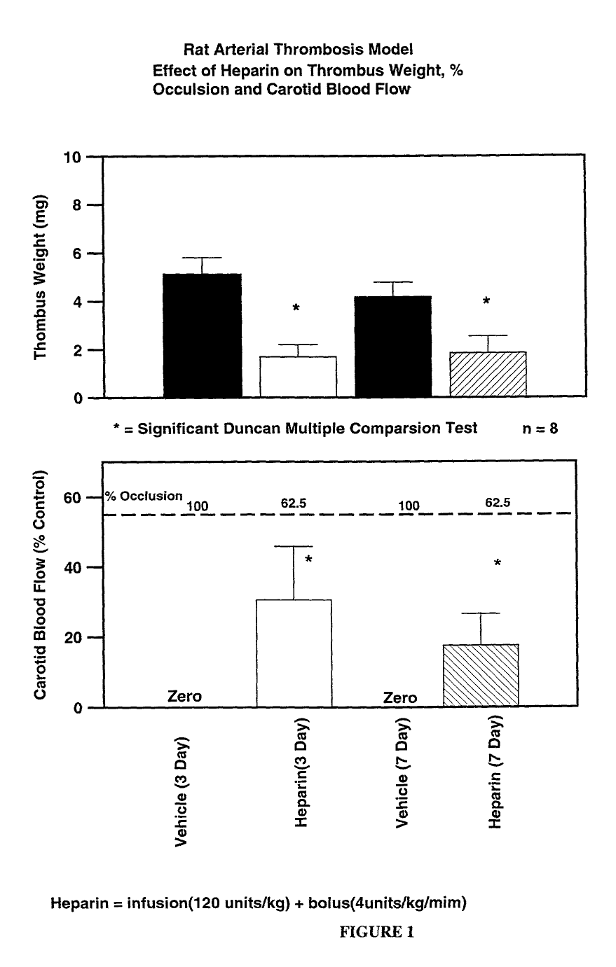

FIGS. 1-3 demonstrate the comparative pharmacology of the anticoagulant/thrombotic regimens by showing the effect of heparin and Factor IX mAb SB249415 on carotid artery occlusion as determined by carotid blood flow. The results indicate that the carotid arteries of all of the vehicle-treated animals occlude in response to the injury.

The results in FIG. 1 demonstrate that heparin given at 120 units bolus with an infusion of 4 units/kg/min at 3 days and 7 days produces a significant decrease in thrombus weight. Carotid blood flow was significantly increased from the vehicle and 62.5% of the animals were occluded.

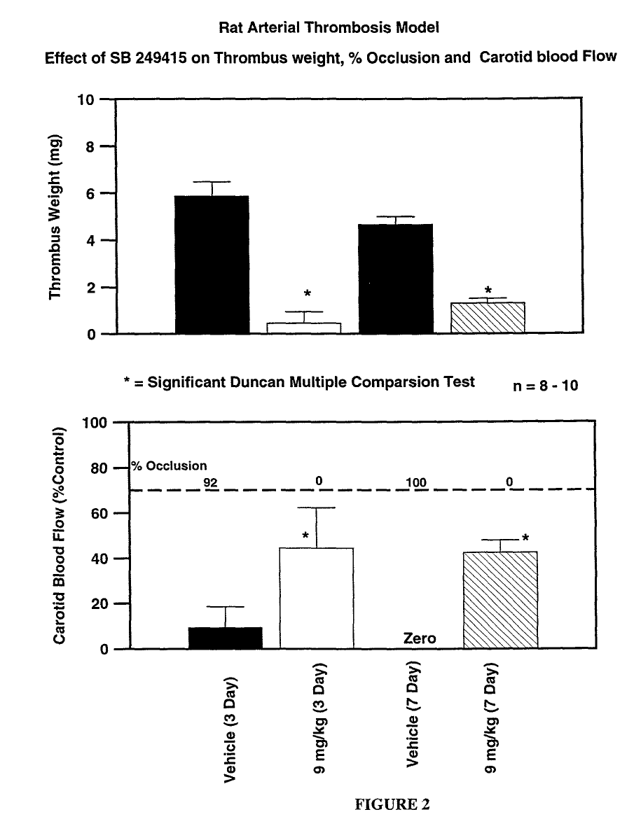

The results in FIG. 2 demonstrate that SB249415 produced an significant decrease in thrombus weight at 3 days and 7 days after dosing with an increase in cartoid blood flow and none of the drug-treated animals occluded.

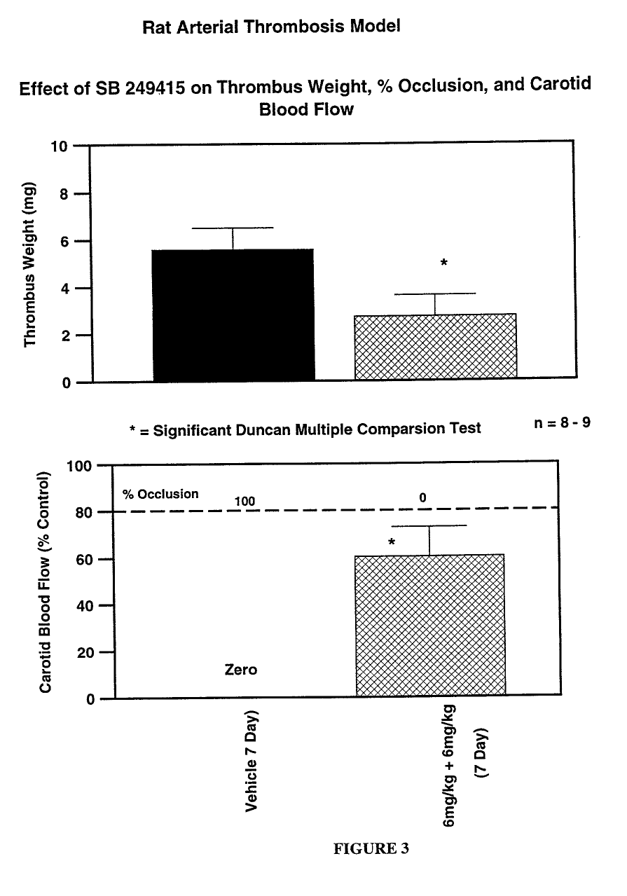

The results in FIG. 3 demonstrate that SB249415 decreased the thrombus weight at doses of 6 mg/kg (i.v.) followed by 6 mg/kg (i.v.) 24 hours later. A significant increase in carotid blood flow with no occlusion was observed in the treated group.

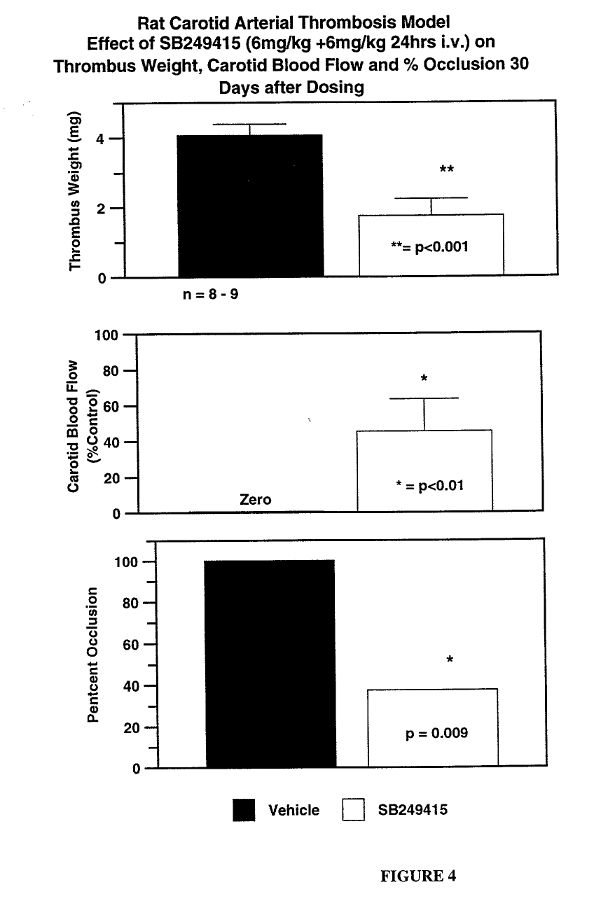

The results in FIG. 4 demonstrate that SB249415 produced a significant decrease in thrombus weigth at 30 days after dosing with an increase in carotid blood flow with no occlusion at dose of 6 mg/kg (i.v.) followed 24 hours later by another 6 mg/kg dose.

The studies conducted in the rat carotid thrombosis model clearly demonstrate a Factor IX mAb duration of efficacy of up to 30 days in prevention of thrombosis in a highly thrombogenic arterial injury model.

### EXAMPLE 2

### Effects of Anti-Factor IX Antibody in Rat Cerebral Artery Thrombus Stroke Model

Studies were carried out to develop and characterize a cerebral thrombus model relevant to focal stroke. Initial work set out to identify the optimum FeCl₃ concentration necessary to completely occlude the middle cerebral artery (MCAO). The MCAO was exposed in anesthetized (65 mg/kg, i.p., pentobarbital) spontaneously hypertensive rats (300-330 gm in weight) via craniotomy under stereotaxic control. A small piece of filter paper (approximately 100 μm²)was soaked in 25%, 12.5% or 6.25% FeCl₃ and placed directly on the MCAO at the level of the inferior cerebral vein for 10 min. Twenty four hr later forelimb and hindlimb neurological deficits were measured in each animal. Rats were then euthanized with an overdose of pentobarbital, forebrains were removed and sliced into seven 2 mm thick coronal sections and stained with triphenyltetrazolium. Percent hemispheric infarct (normalized to the normal contralateral hemisphere) and absolute infarct volume in mm³ were measured in each rat. The resulting data from these treatments indicated that 25% FeCl₃ produced the largest and least variable infarctions and neurological deficits (data not shown). In fact, these values using the 25% solution are practically identical to those produced by electrocoagulation of the MCA at the same level.

Studies were carried out using the 25% FeCl₃-ten min patch application on the MCAO to evaluate if administration of anti-Factor IX monoclonal antibody SB249415 could prevent thrombus formation in the MCA, and thus protect the brain under these conditions.

Fifteen min prior to placing the MCA patch, an i.v. bolus infusion of 3 mg/kg SB249415 was followed by a continuous i.v. infusion of 1 mg/kg over 75 min. Exactly 15 min into this 75 min infusion, the patch was placed on the MCA for 10 min. A different dose of SB249415 administration was made for another group of rats using a 1 mg/kg i.v. bolus followed by a continuous i.v. infusion of 0.3 mg/kg over 75 min. A control group of rats recieved similar volumes of isotonic saline (vehicle) as a bolus i.v. administration followed by a continuous i.v. infusion over 75 min.

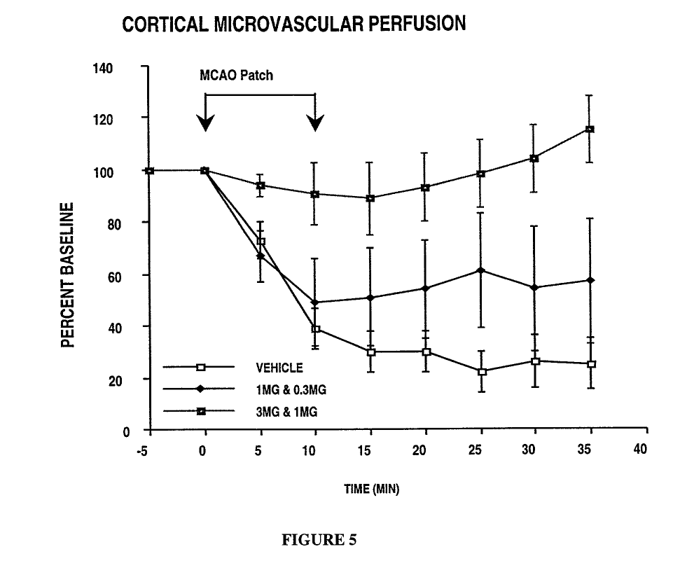

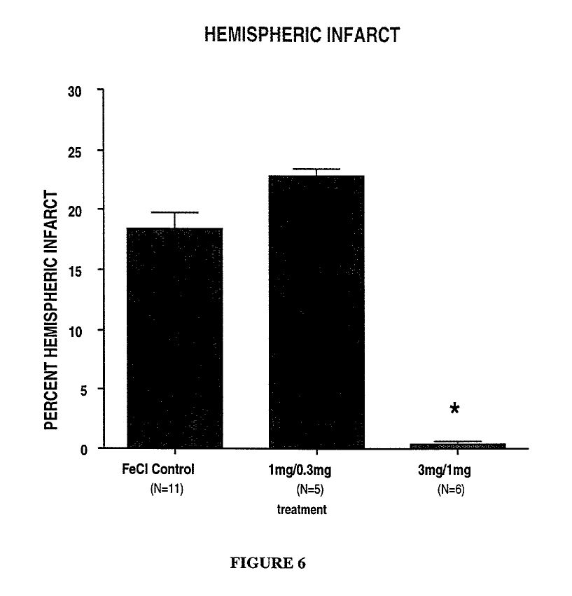

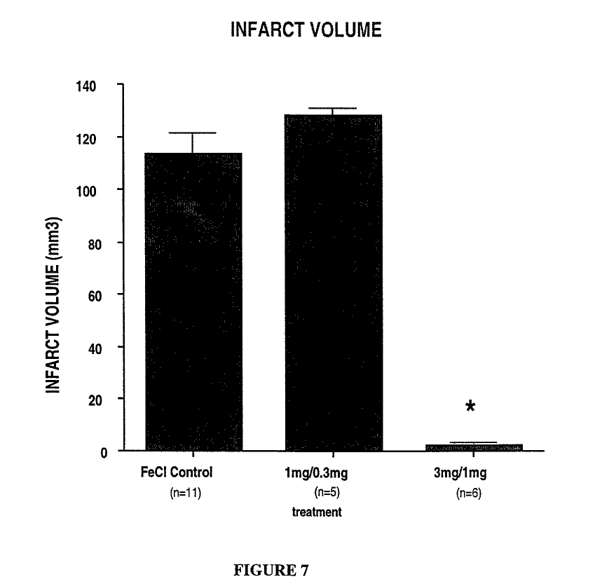

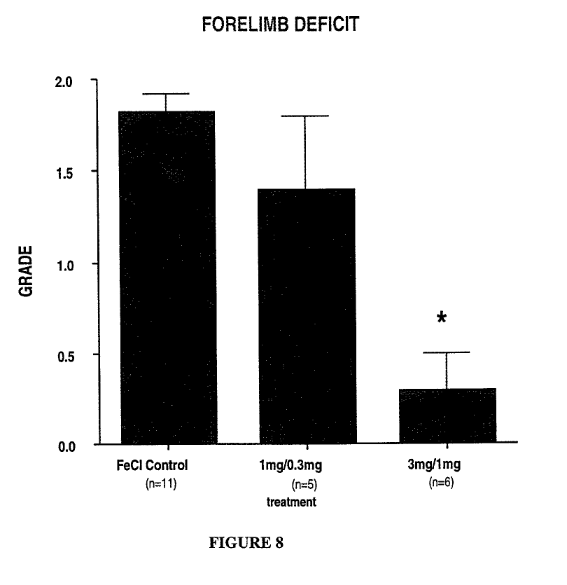

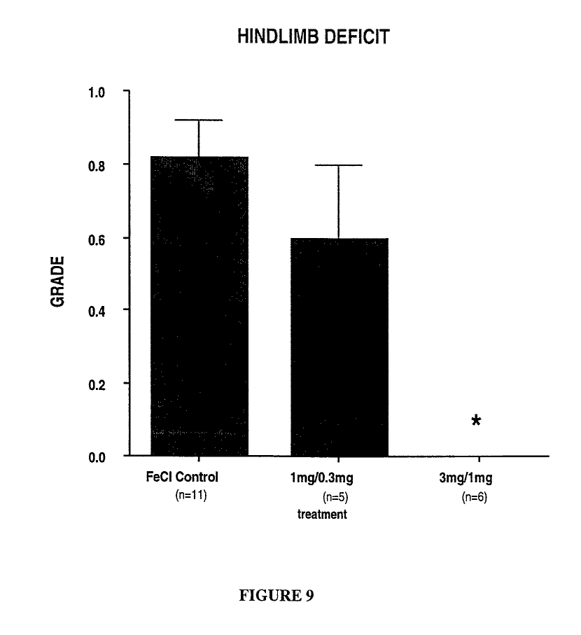

Laser-Doppler Flowmetry was used to record microvascular perfusion in the area receiving blood supply from the MCA for 5 min before and 40 min after the patch was placed on the MCA. Neurological deficits were measured in all animals 24 hr later and brains were removed, sectioned, stained and analyzed as described above. The results are shown in FIGS. 5-9.

Vehicle adminstration did not affect the reduction in cortical microvascular perfusion in the MCA territory, or the production of neurological deficits and brain injury compared to the data presented above. Again, all measures are practically identical to those produced by electrocoagulation of the MCA at the same level. The lower anti-factor IX administration regimen produced variable, non-significant effects on cortical microvascular infusion, and no effects on brain injury or neurological deficits. However, the higher anti-factor IX administration regimen completely eliminated the thrombus formation, the reduced cortical microvascular perfusion, and the brain injury and neurological deficits exhibited by the other two groups (*=p<0.01 compared to all ther groups; ANOVA with Tukey test follow-up analysis). Therefore, the data clearly demonstrate the preventative, protective effects of anti-factor IX in a cerebral arterial thrombosis model relavant to focal ischemic stroke.

The present invention may be embodied in other specific forms without departing from the spirit or essential attributes thereof, and, accordingly, reference should be made to the appended claims, rather than to the foregoing specification, as indicating the scope of the invention.

1. A method for inhibiting thrombosis in an animal comprising administering an effective dose of an anti-coagulation factor antibody or antigen-binding portion thereof having long-lasting protective activity.

2. The method of claim 1 further comprising administering an anti-platelet agent in combination with the anti-coagulation factor monoclonal antibody.

3. The method of claim 2 wherein the anti-platelet agent is acetylsalicylic acid.

4. The method of claim 1 wherein the coagulation factor is from the intrinsic or common coagulation pathway.

5. The method of claim 4 wherein the anti-coagulation factor antibody is an anti-Factor IX, anti-Factor IXa, anti-Factor X, anti-Factor Xa, anti-Factor XI, anti-Factor XIa, anti-Factor VIII, anti-Factor VIIIa, anti-Factor V, anti-Factor Va, anti-Factor VII, anti-Factor VIIa or anti-thrombin.

6. The method of claim 4 wherein the anti-coagulation factor antibody is an anti-Factor IX.

7. The method of claim 6 wherein the anti-Factor IX antibody has the identifying characteristics of SB249413, SB249415, SB249416, SB2249417, SB257731 or SB257732.

8. The method of claim 6 wherein the anti-Factor IX antibody has the identifying characteristics of SB249417.

9. The method of claim 1 wherein the long-lasting protective activity has a duration of up to 3 days.

10. The method of claim 1 wherein the long-lasting protective activity has a duration of up to 7 days.

11. The method of claim 1 wherein the long-lasting protective activity has a duration of up to 30 days.

12. The method of claim 1 wherein the thrombosis is associated with myocardial infarction, unstable angina, atrial fibrillation, stroke, renal damage, pulmonary embolism, deep vein thrombosis, percutaneous translumenal coronary angioplasty, disseminated intravascular coagulation, sepsis, artificial organs, shunts or prostheses.
